(19) United States
(12) Patent Application Publication
(10) Pub. No.: US 2009/0306479 A1
(43) Pub. Date: Dec. 10, 2009

(54) VARIABLE SPECTROSCOPY ELEMENT, SPECTROSCOPY APPARATUS, AND ENDOSCOPE SYSTEM

(75) Inventor: Yasuhiro Kamihara, Tokyo (JP)

Correspondence Address: SCULLY, SCOTT, MURPHY & PRESSER, P.C., 400 GARDEN CITY PLAZA, SUITE 300, GARDEN CITY, NY, 11530, US

(73) Assignee: OLYMPUS CORPORATION, Tokyo (JP)

(21) Appl. No.: 12/518,786

(22) PCT Filed: Dec. 14, 2007
(86) PCT No.: PCT/JP2007/074114

§ 371 Date: Jun. 11, 2009

(30) Foreign Application Priority Data
Dec. 14, 2006 (JP) 2006-337595

(51) Int. Cl. A61B 1/06; G01B 9/02

(52) U.S. Cl. 600/180; 356/454; 600/178

(57) ABSTRACT

Compactness and easier assembly, as well as desired spectral characteristics, are achieved without requiring an accurate assembly process, by accurately detecting the spacing between optical substrates. A variable spectroscopy element (1) is provided, which includes two optical substrates (4a and 4b) that face each other with a spacing therebetween; optical coatings (3) provided on opposing surfaces of the optical substrates (4a and 4b); an actuator (4c) that adjusts the spacing between the two optical substrates (4a and 4b); and a capacitance sensor (6) that has sensor electrodes (6a and 6b) respectively provided on the two optical substrates (4a and 4b) and detects the spacing between the optical substrates (4a and 4b). The sensor electrode (6b) provided on one optical substrate (4b) is included within a region of the optical substrate (4b) onto which the sensor electrode (6a) provided on the other optical substrate (4a) is projected.

### TECHNICAL FIELD

The present invention relates to variable spectroscopy elements, spectroscopy apparatuses, and endoscope systems.

### BACKGROUND ART

In known etalon-type variable spectroscopy elements, two optical substrates provided with optical coatings on opposing surfaces thereof are disposed facing each other and a spacing therebetween is adjustable by means of an actuator formed of a piezoelectric element (for example, see Patent Document 1).

Such a variable spectroscopy element has sensor electrodes of a capacitance sensor provided on the opposing surfaces of the two optical substrates and detects the spacing between the optical substrates with the capacitance sensor so as to control the spacing while maintaining parallelism.

Patent Document 1: Japanese Unexamined Patent Application, Publication No. Hei 1-94312

### DISCLOSURE OF INVENTION

The present invention provides a variable spectroscopic element, a spectroscopy apparatus, and an endoscope system that are compact and enable easier assembly and can achieve desired spectral characteristics, without requiring an accurate assembly process, by accurately detecting the spacing between the optical substrates.

A first aspect of the present invention provides a variable spectroscopy element that includes optical coatings provided on opposing surfaces of first and second optical substrates that face each other with a spacing therebetween; an actuator that adjusts the spacing between the first and second optical substrates; a first sensor electrode that detects the spacing between the first and second optical substrates and is provided on the first optical substrate; and a second sensor electrode that detects the spacing between the first and second optical substrates, the second sensor electrode facing the first sensor electrode and provided within a region of the second optical substrate onto which the first sensor electrode is projected.

In the first aspect of the present invention, the first and second sensor electrodes may have a similar shape.

In the first aspect of the present invention, the first and second sensor electrodes may be circular.

In the first aspect of the present invention, the optical coatings may be composed of a conductive material, and the first and second sensor electrodes may be formed of the optical coatings.

In the first aspect of the present invention, the first and second sensor electrodes may have different shapes.

In the first aspect of the present invention, the first and second sensor electrodes may have a dimensional difference that is greater in a circumferential direction than in a radial direction.

In the first aspect of the present invention, the optical coatings may transmit light of a desired wavelength range.

A second aspect of the present invention provides a spectroscopy apparatus that includes the aforementioned variable spectroscopy element and an image-acquisition unit that acquires an image of light split by the variable spectroscopy element.

A third aspect of the present invention provides an endoscope system that includes the aforementioned variable spectroscopy apparatus.

The present invention can advantageously achieve compactness and easier assembly, as well as desired spectral characteristics, without requiring an accurate assembly process, by accurately detecting the spacing between optical substrates.

### BRIEF DESCRIPTION OF DRAWINGS

FIG. 1 is a longitudinal sectional view showing an image-acquisition unit equipped with a variable spectroscopy element according to an embodiment of the present invention.

FIG. 2 illustrates an arrangement example of reflective films and sensor electrodes when optical substrates of the variable spectroscopy element shown in FIG. 1 are viewed from an optical-axis direction.

FIG. 3 illustrates a first modification of the sensor electrodes in the variable spectroscopy element shown in FIG. 2.

FIG. 4 illustrates a second modification of the sensor electrodes in the variable spectroscopy element shown in FIG. 2.

FIG. 5 illustrates a third modification of the sensor electrodes in the variable spectroscopy element shown in FIG. 2.

FIG. 6 illustrates a fourth modification of the sensor electrodes in the variable spectroscopy element shown in FIG. 2.

FIG. 7 illustrates a fifth modification of the sensor electrodes in the variable spectroscopy element shown in FIG. 2.

FIG. 8 illustrates a sixth modification of the sensor electrodes in the variable spectroscopy element shown in FIG. 2.

FIG. 9 illustrates the overall configuration of an endoscope system according to an embodiment of the present invention.

FIG. 10 illustrates transmittance characteristics of a variable spectroscopy element constituting an image-acquisition unit provided in the endoscope system shown in FIG. 9.

FIG. 11 is a timing chart explaining the operation of the endoscope system shown in FIG. 9.

FIG. 12 illustrates an electrical circuit that amplifies a signal from sensors in the variable spectroscopy element constituting the image-acquisition unit provided in the endoscope system shown in FIG. 9.

FIG. 13 illustrates an example of the electrical circuit when the variable spectroscopy element shown in FIG. 7 is used.

FIG. 14 illustrates a modification of the endoscope system shown in FIG. 9 and is a longitudinal sectional view showing an example of a light source unit disposed at the tip of an insertion section.

### EXPLANATION OF REFERENCE SIGNS

1: variable spectroscopy element

3: reflective film (optical coating)

4a, 4b: optical substrate

4c: actuator

6: sensor (capacitance sensor)

6a, 6b: sensor electrode

10: endoscope system (spectroscopy apparatus)

21: image-acquisition element

### BEST MODE FOR CARRYING OUT THE INVENTION

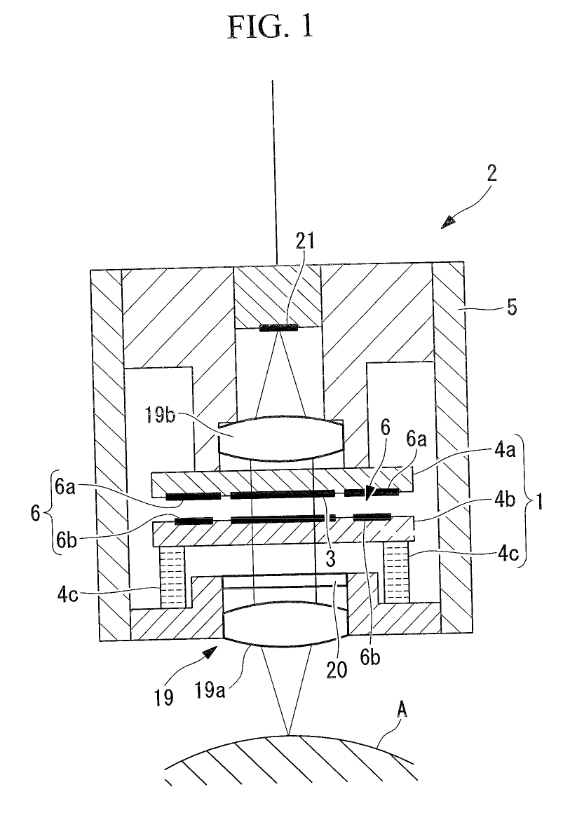

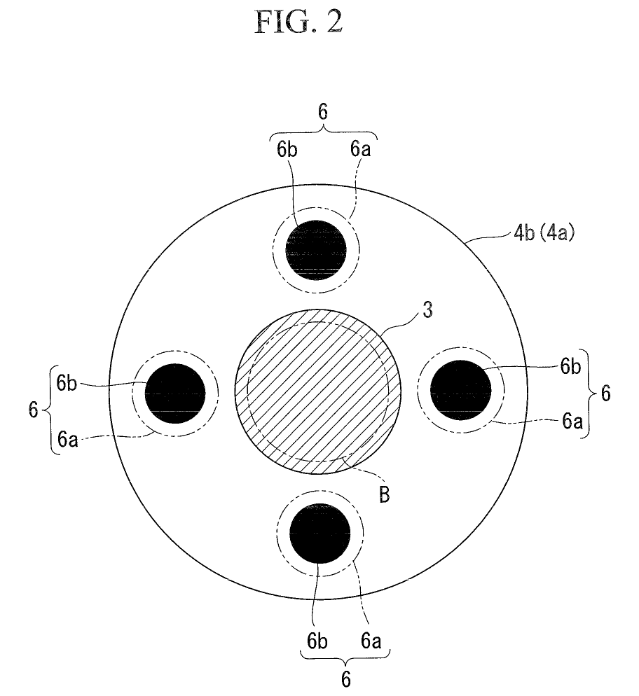

A variable spectroscopy element 1 according to a first embodiment of the present invention will be described below with reference to FIGS. 1 and 2.

As shown in FIG. 1, the variable spectroscopy element 1 according to this embodiment is included in an image-acquisition unit 2 and is an etalon-type optical filter that includes two circular optical substrates 4a and 4b, disposed substantially in parallel to each other with a spacing therebetween and respectively having reflective films (optical coatings) 3 on opposing surfaces thereof, and actuators 4c that adjust the spacing between the optical substrates 4a and 4b. The optical substrate 4a is directly fixed to a frame member 5 constituting the image-acquisition unit 2, whereas the optical substrate 4b is attached to the frame member 5 with the actuators 4c therebetween.

The actuators 4c are multilayer piezoelectric elements and are provided at four locations, which are spaced at equal distances in the circumferential direction, around the edge of the optical substrate 4b.

The variable spectroscopy element 4 actuates the actuators 4c so as to adjust the spacing between the optical substrates 4a and 4b. By adjusting the spacing between the optical substrates 4a and 4b in this manner, the variable spectroscopy element 1 can change the wavelength range of light passing therethrough in the axis direction.

The two optical substrates 4a and 4b constituting the variable spectroscopy element 1 are provided with sensors 6 for detecting the spacing between the optical substrates 4a and 4b. The sensors 6 are of a capacitance type and include a plurality of sensor electrodes 6a and 6b provided at opposing positions in outer peripheral areas, which are located outside an effective optical diameter B (see FIG. 2), of the optical substrates 4a and 4b. The sensor electrodes 6a and 6b are disposed at four locations in the outer peripheral areas of the optical substrates 4a and 4b and are spaced at equal distances in the circumferential direction. Metallic films may be used as the sensor electrodes 6a and 6b.

The capacitance sensors 6 are configured to utilize a characteristic in which the capacitance between the sensor electrodes 6a and 6b varies in inverse proportion to the spacing therebetween, and to detect the spacing between the optical substrates 4a and 4b on the basis of the magnitude of the capacitance between the sensor electrodes 6a and 6b.

In the variable spectroscopy element 1 according to this embodiment, the sensor electrodes 6a and 6b both have a circular shape, as shown in FIG. 2. As shown in FIGS. 1 and 2, of the sensor electrodes 6a and 6b, the sensor electrodes 6a provided on one optical substrate 4a have a radius larger than that of the sensor electrodes 6b provided on the other optical substrate 4b. Moreover, as shown in FIG. 2, the sensor electrodes 6b provided on the optical substrate 4b are each disposed within a region (i.e., a region indicated by a dashed line) of the optical substrate 4b onto which the corresponding sensor electrode 6a provided on the optical substrate 4a is projected, as viewed in the optical-axis direction.

In fluorescence observation, the transmission efficiency of an optical system is extremely important since the fluorescence intensity obtained from an observation object is generally weak. Although high transmittance can be obtained in the etalon-type variable spectroscopy element 1 when the reflective films are parallel to each other, the transmittance is significantly lowered if there is a parallelism error. Therefore, in order to correct a tilt error occurring in the two optical substrates 4a and 4b when the spacing therebetween is adjusted, the variable spectroscopy element 1 used in the image-acquisition unit 2 for fluorescence observation is preferably provided with a plurality of sensors 6 so as to have multiple degrees of freedom.

The variable spectroscopy element 1 according to this embodiment performs feedback control on a drive signal sent to the actuators 4c on the basis of a signal received from the sensor electrodes 6a and 6b so as to improve the accuracy in the control of the transmittance characteristics.

The operation of the variable spectroscopy element 1 according to this embodiment having the above configuration will be described below.

In the variable spectroscopy element 1 according to this embodiment, light is made to enter an area of the effective optical diameter B of the two optical substrates 4a and 4b disposed parallel to each other with a spacing therebetween, so that only a portion of the light having a wavelength determined in accordance with the spacing between the optical substrates 4a and 4b is transmitted through the two optical substrates 4a and 4b, whereas the remaining portion of the light is reflected. By actuating the actuators 4c, the spacing between the two optical substrates 4a and 4b can be adjusted, thereby changing the wavelength of light to be transmitted through the two optical substrates 4a and 4b. By adjusting the spacing between the two optical substrates 4a and 4b in this manner, light of a desired wavelength range to be observed can be split off from light of other wavelength ranges.

The opposing surfaces of the optical substrates 4a and 4b respectively have the sensor electrodes 6a and 6b disposed thereon in a face-to-face manner. Thus, a voltage signal indicating the capacitance formed between the sensor electrodes 6a and 6b is detected by the sensor electrodes 6a and 6b, whereby the spacing between the sensor electrodes 6a and 6b can be detected in accordance with the voltage signal. Since the sensor electrodes 6a and 6b are provided in four pairs in the circumferential direction of the optical substrates, each pair of sensor electrodes 6a and 6b can detect the spacing between the optical substrates 4a and 4b at a corresponding position. By controlling the actuators 4c on the basis of the spacing detected in this manner, the spacing can be accurately adjusted while maintaining the two optical substrates 4a and 4b in a parallel state.

In this case, in the variable spectroscopy element 1 according to this embodiment, the opposing sensor electrodes 6a and 6b have different radii. Therefore, an opposing area equivalent to the area of the smaller sensor electrodes 6b can be obtained without having to perform an accurate positioning process during assembly. In other words, in this variable spectroscopy element 1, the sensor electrodes 6b provided on one optical substrate 4b are each disposed within the region of the optical substrate 4b onto which the corresponding sensor electrode 6a provided on the other optical substrate 4a is projected. Therefore, in this variable spectroscopy element 1, even if the two optical substrates 4a and 4b are assembled in a slightly misaligned state in a direction perpendicular to the thickness direction thereof, namely, the radial direction or the circumferential direction of the optical substrates 4a and 4b, there is no change in the capacitance formed between the sensor electrodes 6a and 6b.

By driving the plurality of actuators 4c, the spacing between the two optical substrates 4a and 4b can be accurately adjusted. It is conceivable that individual differences in the actuators 4c may cause the two optical substrates 4a and 4b to be positionally misaligned with respect to each other in the direction perpendicular to the thickness direction. Even in that case, there is no change in the capacitance formed between the sensor electrodes 6a and 6b.

Accordingly, a voltage signal indicating the capacitance that uniquely corresponds to the spacing between the two optical substrates 4a and 4b can be detected, and the spacing between the two optical substrates 4a and 4b can be accurately controlled on the basis of the voltage signal, thereby advantageously allowing for accurate splitting of light of a desired wavelength range.

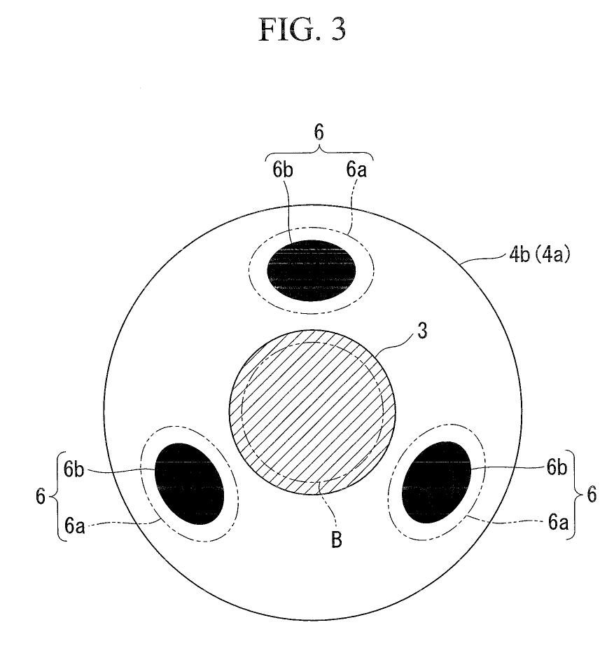

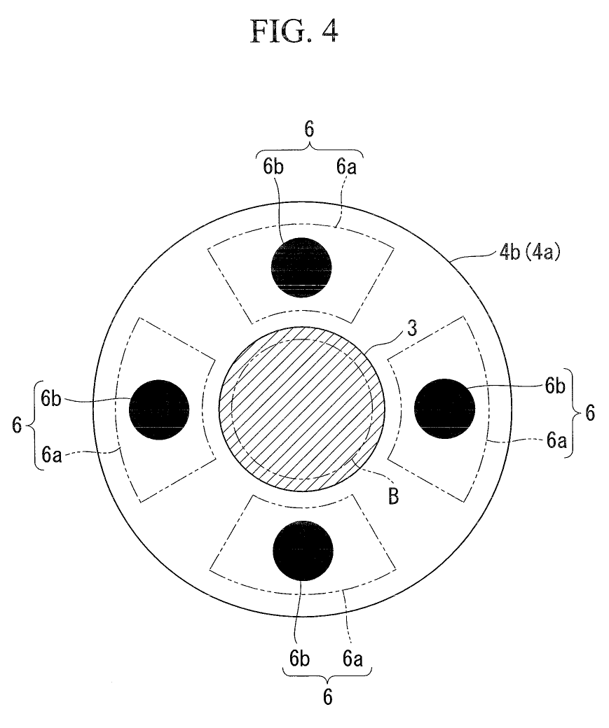

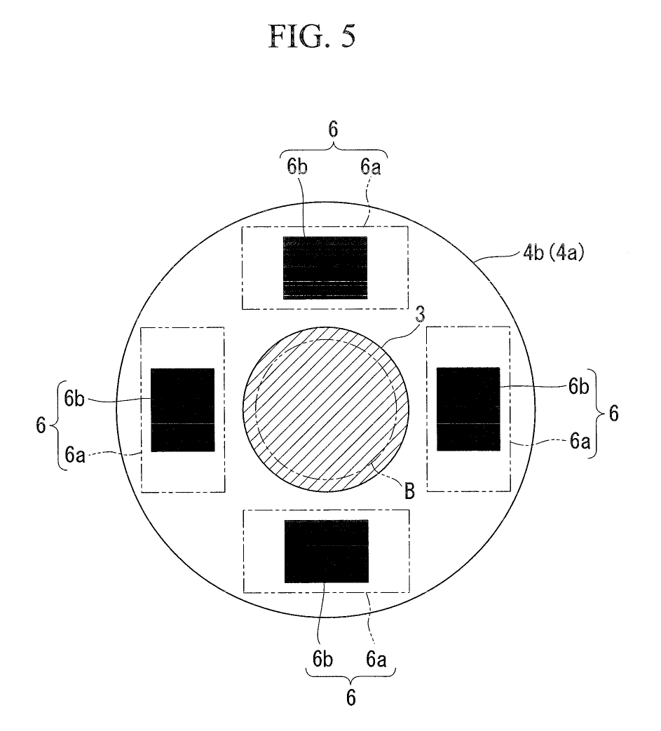

In the variable spectroscopy element 1 according to this embodiment, the sensor electrodes 6a and 6b are provided in four pairs in the circumferential direction of the optical substrates 4a and 4b. Alternatively, as shown in FIG. 3, the sensor electrodes 6a and 6b may be provided in three pairs, or a desired number thereof may be provided. In the example shown in FIG. 3, the sensor electrodes 6a and 6b used have an elliptical shape. The shape thereof is not particularly limited, and a freely chosen shape may be used, such as a sector shape or a rectangular shape, as shown in FIG. 4 or FIG. 5.

In that case, it is preferable that the sensor electrodes 6a and 6b in FIGS. 4 and 5 have shapes such that the larger sensor electrodes 6a have a dimensional difference in the circumferential direction greater than a dimensional difference in the radial direction relative to the smaller sensor electrodes 6b. The circular optical substrates 4a and 4b can be positioned substantially accurately with respect to each other in the radial direction by aligning the outer peripheries thereof, as viewed in the optical axis direction. However, it is difficult to position the optical substrates 4a and 4b with respect to each other in the circumferential direction. By giving a large dimensional difference between the sensor electrodes 6a and 6b in the circumferential direction, the capacitance detected by the sensor electrodes 6a and 6b can be prevented from changing even if the optical substrates 4a and 4b are roughly positioned with respect to each other in the circumferential direction, thereby advantageously facilitating the assembly process.

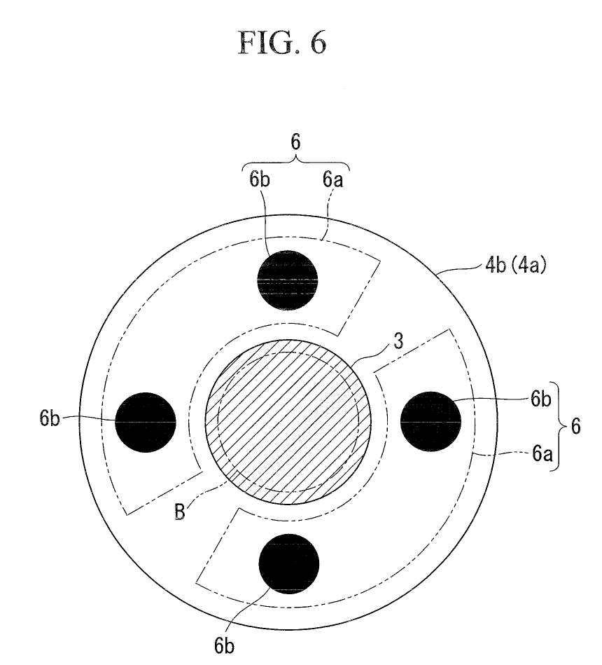

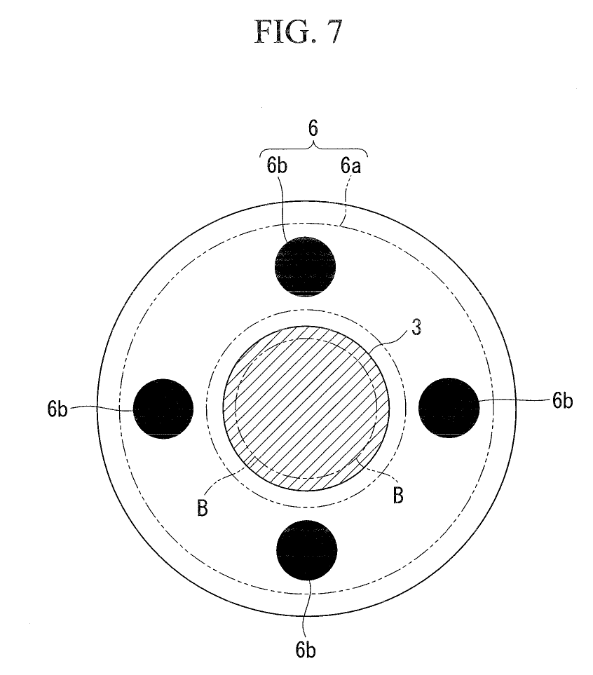

As shown in FIGS. 6 and 7, the number of sensor electrodes 6a and 6b respectively provided on the optical substrates 4a and 4b does not necessarily need to be the same between the two. Specifically, as shown in FIG. 6, for every two sensor electrodes 6b provided on one optical substrate 4b and spaced apart by a certain distance in the circumferential direction, a single sensor electrode 6a with a size that can face both of these two sensor electrodes 6b may be provided on the other optical substrate 4a. Alternatively, as shown in FIG. 7, for multiple sensor electrodes 6b provided on one optical substrate 4b and spaced apart by a certain distance in the circumferential direction, a single ring-shaped sensor electrode 6a that faces all of these sensor electrodes 6b may be provided on the other optical substrate 4a.

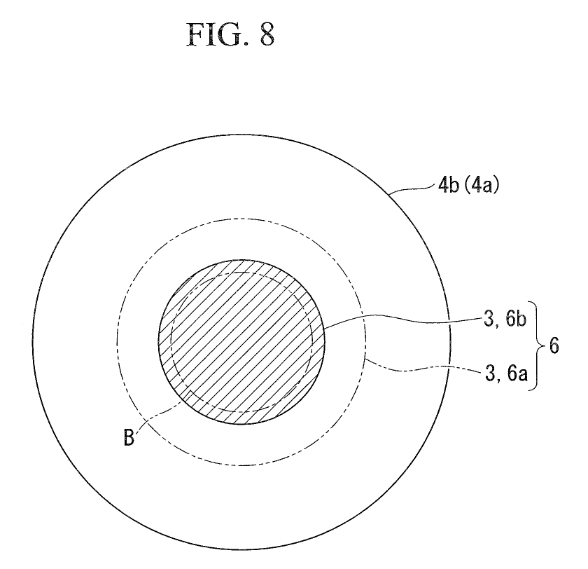

In an example shown in FIG. 8, the reflective films 3 provided on the opposing surfaces of the optical substrates 4a and 4b may be composed of a conductive material so that the reflective films 3 themselves can also serve as the sensor electrodes 6a and 6b for forming a capacitance. In that case, it is preferable that circular reflective films 3 having different radii be provided in the center of the respective optical substrates 4a and 4b. With this configuration, even if the optical substrates 4a and 4b are assembled in a misaligned state in the radial direction or the circumferential direction or become misaligned with respect to each other in one of these directions due to actuation of the actuators 4c, the same voltage signal can be output so long as the spacing is the same, thereby improving the detection accuracy. Alternatively, reflective films 3 having the same radii may be provided in the center of the optical substrates 4a and 4b so that these reflective films 3 can also serve as the sensor electrodes 6a and 6b. This is advantageous in that the detection accuracy of the spacing between the optical substrates 4a and 4b can be prevented from being reduced when the optical substrates 4a and 4b are positionally misaligned with respect to each other in the circumferential direction.

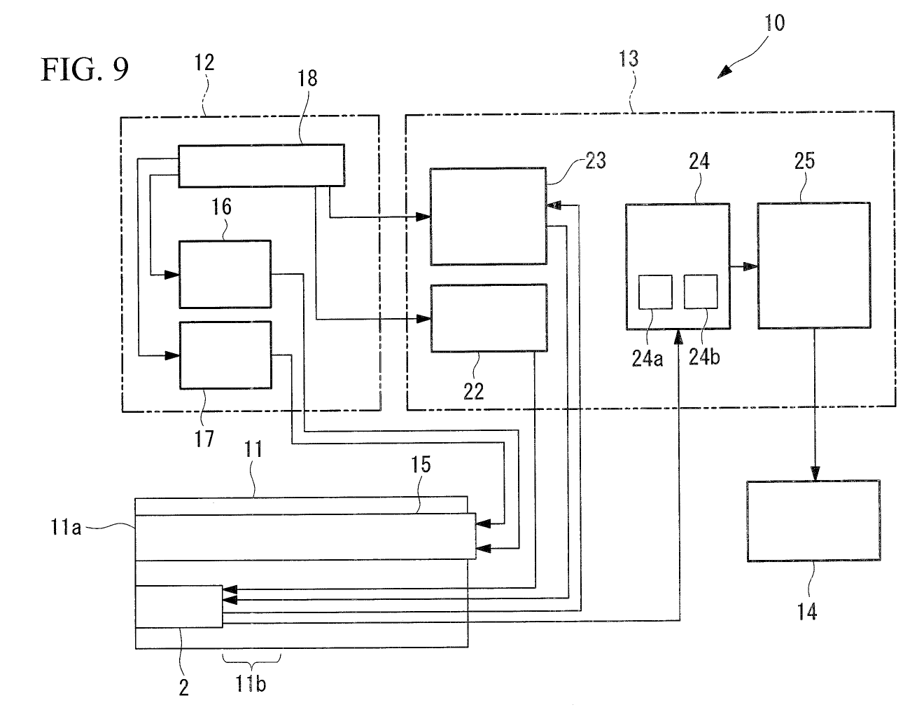

An endoscope system 10 according to an embodiment of the present invention will now be described with reference to FIGS. 9 to 12.

As shown in FIG. 9, the endoscope system 10 according to this embodiment includes an insertion section 11 to be inserted into a body cavity of a living organism, an image-acquisition unit 2 disposed inside the insertion section 11, a light source unit 12 that emits various kinds of light, a control unit 13 that controls the image-acquisition unit 2 and the light source unit 12, and a display unit 14 that displays an image acquired by the image-acquisition unit 2.

The insertion section 11 has an extremely narrow dimension so that it can be inserted into a body cavity of a living organism. The insertion section 11 contains the image-acquisition unit 2 and a light guide 15 that transmits the light from the light source unit 12 to a tip 11a.

The light source unit 12 includes an illumination light source 16 that emits illumination light to illuminate an observation object A inside the body cavity so that reflected light returning from the observation object A can be obtained, an excitation light source 17 that emits excitation light to the observation object A inside the body cavity to excite a fluorescent material existing in the observation object A so that fluorescence can be produced, and a light-source control circuit 18 that controls these light sources 16 and 17.

The illumination light source 16 is a combination of, for example, a xenon lamp and a band-pass filter (not shown), and a 50% transmission range of the band-pass filter is from 430 nm to 460 nm. In other words, the light source 16 is configured to generate illumination light in a wavelength range of 430 nm to 460 nm.

The excitation light source 17 is, for example, a semiconductor laser that emits excitation light with a peak wavelength of 660±5 nm. Excitation light with this wavelength can excite fluorescent agents, such as Cy5.5 (manufactured formerly by Amersham Inc. but currently by GE Health Care Inc.) and Alexa Fluor 700 (manufactured by Molecular Probes Inc.)

The light-source control circuit 18 is configured to alternately turn on and off the illumination light source 16 and the excitation light source 17 at predetermined timings based on a timing chart to be described later.

The image-acquisition unit 2 is disposed at an end portion of the insertion section 11. The end portion of the insertion section 11 is located closer towards the tip 11a relative to the center of the insertion section 11 in the lengthwise direction thereof, and preferably, is located closer towards the tip 11a relative to a bending portion 11b that can be bent for changing the orientation of the tip 11a of the insertion section 11.

As shown in FIG. 1, the image-acquisition unit 2 includes an image-acquisition optical system 19 including lenses 19a and 19b that collect light received from the observation object A, an barrier filter 20 that blocks excitation light received from the observation object A, the aforementioned variable spectroscopy element 1 whose spectral characteristics can be varied by the operation of the control unit 13, an image-acquisition element 21 that acquires an image of the light collected by the image-acquisition optical system 19 and converts it into an electrical signal, and the frame member 5 that supports these components.

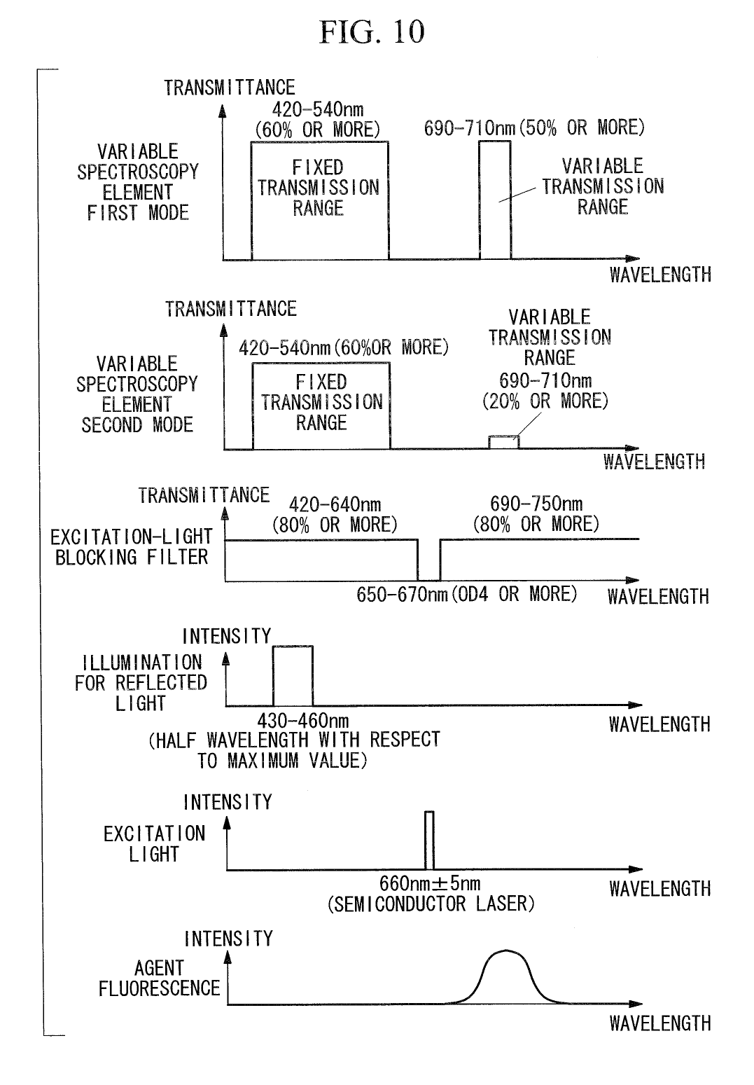

In further detail, as shown in FIG. 10, the variable spectroscopy element 1 has a transmittance-versus-wavelength characteristic having two transmission ranges, namely, one fixed transmission range and one variable transmission range. In the fixed transmission range, incident light is constantly transmitted regardless of the state of the variable spectroscopy element 1. On the other hand, in the variable transmission range, the transmittance characteristics vary depending on the state of the variable spectroscopy element 1.

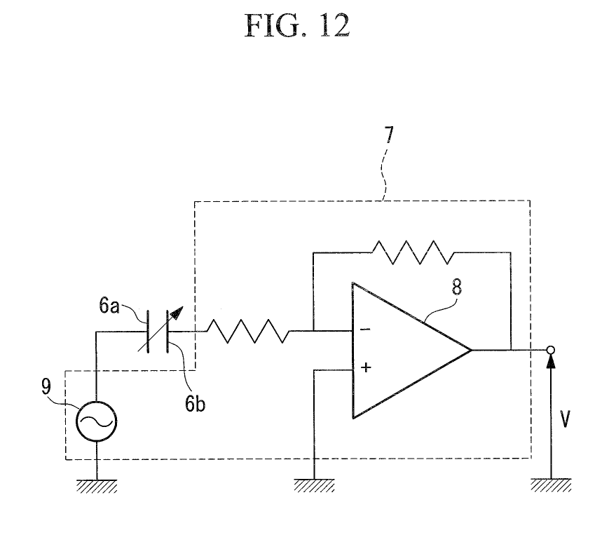

The sensor electrodes 6a and 6b are connected to, for example, an electrical circuit 7, as shown in FIG. 12. The electrical circuit 7 supplies alternating current to the sensor electrodes 6a and 6b, converts the capacitance between the sensor electrodes 6a and 6b, determined according to the spacing between the optical substrates 4a and 4b, to an electrical signal, amplifies the electrical signal, and outputs a voltage V. In FIG. 12, a component denoted by reference numeral 8 is an operational amplifier, which is an active element, and a component denoted by reference numeral 9 is an AC power supply. The electrical circuit 7 is fixed to the optical substrate 4a, which is fixed to the frame member 5.

In fluorescence observation, the transmission efficiency of an optical system is extremely important since the fluorescence intensity obtained from an observation object is generally weak. Although high transmittance can be obtained in the etalon-type variable spectroscopy element 1 when the reflective films are parallel to each other, the transmittance is significantly lowered if there is a parallelism error. Therefore, in order to correct a tilt error occurring in the two optical substrates 4a and 4b when the spacing therebetween is adjusted, the variable spectroscopy element 1 used in the image-acquisition unit 2 for fluorescence observation is preferably provided with a plurality of sensors 6 so as to have multiple degrees of freedom.

The endoscope system 10 according to this embodiment performs feedback control on a drive signal sent to the actuators 4c on the basis of a signal received from the sensor electrodes 6a and 6b so as to improve the accuracy in the control of the transmittance characteristics.

As shown in FIG. 9, the control unit 13 includes an image-acquisition-element drive circuit 22 that controls the driving of the image-acquisition element 21, a variable spectroscopy-element control circuit 23 that controls the driving of the variable spectroscopy element 1, a frame memory 24 that stores image information acquired by the image-acquisition element 21, and an image processing circuit 25 that processes the image information stored in the frame memory 24 and outputs it to the display unit 14.

The image-acquisition-element drive circuit 22 and the variable spectroscopy-element control circuit 23 are connected to the light-source control circuit 18 and control the driving of the variable spectroscopy element 1 and the image-acquisition element 21 in synchronization with a switching operation between the illumination light source 16 and the excitation light source 17 performed by the light-source control circuit 18.

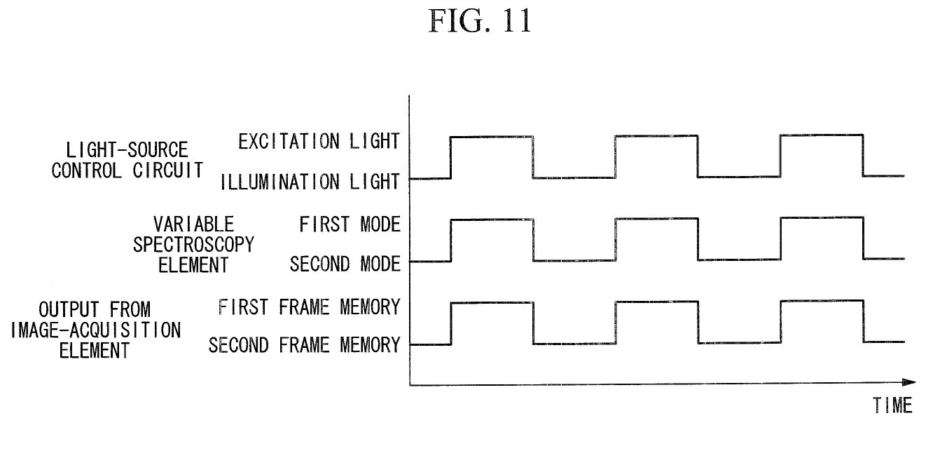

In detail, as shown in a timing chart in FIG. 11, when the light-source control circuit 18 is actuated to cause the excitation light source 17 to emit excitation light, the variable spectroscopy-element control circuit 23 sets the variable spectroscopy element 1 in a first mode in which the image-acquisition-element drive circuit 22 is made to output image information, output from the image-acquisition element 21, to a first frame memory 24a. On the other hand, when illumination light is emitted from the illumination light source 16, the variable spectroscopy-element control circuit 23 sets the variable spectroscopy element 1 in a second mode in which the image-acquisition-element drive circuit 22 is made to output image information, output from the image-acquisition element 21, to a second frame memory 24b.

The image processing circuit 25 is configured to, for example, receive fluorescence image information, acquired as the result of the emission of the excitation light, from the first frame memory 24a and output it on a first channel of the display unit 14, and is also configured to receive reflection image information, acquired as the result of the emission of the illumination light, from the second frame memory 24b and output it on a second channel of the display unit 14.

The operation of the endoscope system 10 according to this embodiment having the above configuration will be described below.

When an image of the observation object A inside a body cavity of a living organism is to be acquired by using the endoscope system 10 according to this embodiment, a fluorescent agent is injected into the body and the insertion section 11 is inserted into the body cavity so that the tip 11a thereof is made to face the observation object A inside the body cavity. In this state, the light source unit 12 and the control unit 13 are actuated so as to actuate the light-source control circuit 18, thereby alternately actuating the illumination light source 16 and the excitation light source 17 to cause them to generate illumination light and excitation light, respectively.

The excitation light and the illumination light generated in the light source unit 12 are transmitted to the tip 11a of the insertion section 11 via the light guide 15 and are emitted from the tip 11a of the insertion section 11 towards the observation object A.

When the excitation light is emitted to the observation object A, the fluorescent agent existing in the observation object A is excited and thus emits fluorescence. The fluorescence emitted from the observation object A is transmitted through the lens 19a and the barrier filter 20 in the image-acquisition unit 2 so as to enter the variable spectroscopy element 1.

Since the variable spectroscopy element 1 is switched to the first mode, by the actuation of the variable spectroscopy-element control circuit 23, in synchronization with the actuation of the excitation light source 17, the variable spectroscopy element 1 has higher transmittance for the fluorescence and can thus transmit the incident fluorescence. In this case, a portion of the excitation light emitted to the observation object A is reflected by the observation object A and enters the image-acquisition unit 2 together with the fluorescence. However, because the image-acquisition unit 2 is provided with the barrier filter 20, the excitation light is blocked and prevented from entering the image-acquisition element 21.

The fluorescence transmitted through the variable spectroscopy element 1 enters the image-acquisition element 21 where fluorescence image information is acquired. The acquired fluorescence image information is stored in the first frame memory 24a and is output on the first channel of the display unit 14 by the image processing circuit 25 so as to be displayed by the display unit 14.

On the other hand, when the illumination light is emitted to the observation object A, the illumination light is reflected off the surface of the observation object A. This illumination light is transmitted through the lens 19a and the barrier filter 20 so as to enter the variable spectroscopy element 1. Since the wavelength range of the reflected light of the illumination light is located in the fixed transmission range of the variable spectroscopy element 1, the reflected light received by the variable spectroscopy element 1 is entirely transmitted through the variable spectroscopy element 1.

The reflected light transmitted through the variable spectroscopy element 1 enters the image-acquisition element 21 where reflection image information is acquired. The acquired reflection image information is stored in the second frame memory 24b and is output on the second channel of the display unit 14 by the image processing circuit 25 so as to be displayed by the display unit 14.

In this case, because the excitation light source 17 is turned off, fluorescence is not produced by excitation light having a wavelength of 660 nm. Because the wavelength range of the illumination light source 16 has extremely low excitation efficiency for the fluorescent agent, it can be considered that there is substantially nothing produced. In addition, since the variable spectroscopy element 1 is switched to the second mode, by the actuation of the variable spectroscopy-element control circuit 23, in synchronization with the actuation of the illumination light source 16, the variable spectroscopy element 1 has lower transmittance for the fluorescence and thus blocks the fluorescence even when it is incident thereon. Accordingly, only an image of the reflected light is acquired by the image-acquisition element

Consequently, with the endoscope system 10 according to this embodiment, a fluorescence image and a reflection image can be provided to the user.

In this case, in the endoscope system 10 according to this embodiment, because the sensors 6 are provided in the variable spectroscopy element 1, the sensors 6 can detect the spacing between the two optical substrates 4a and 4b, and feedback control can be performed on the voltage signal applied to the actuators 4c when performing the switching operation between the first mode and the second mode. Consequently, the spacing between the optical substrates 4a and 4b can be accurately controlled so as to allow for accurate splitting of light of a desired wavelength range, whereby a sharp fluorescence image and a sharp reflection image can be acquired.

Furthermore, in this embodiment, the electrical signal output from the sensor electrodes 6a and 6b and indicating the capacitance between the sensor electrodes 6a and 6b is amplified by the electrical circuit 7, fixed to the optical substrate 4b of the variable spectroscopy element 1, and is reduced in output impedance. Subsequently, the electrical signal is transmitted to the insertion section 11 and is then sent from the base end of the insertion section 11 to the variable spectroscopy-element control circuit 23 outside the body. In consequence, mixing of noise into the electrical signal detected by the sensors 6 can be reduced, and the spacing between the optical substrates 4a and 4b can be accurately detected, whereby the spectral characteristics of the variable spectroscopy element 1 can be advantageously controlled with high accuracy.

In this embodiment, the sensor electrodes 6a and 6b provided on the opposing surfaces of the respective optical substrates 4a and 4b have different outside dimensions. Therefore, in this embodiment, when the actuators 4c are driven, even if misalignment occurs between the optical substrates 4a and 4b in the direction perpendicular to the optical axis due to individual differences in the actuators 4c, there is no change in the capacitance formed between the opposing sensor electrodes 6a and 6b, and the spacing between the optical substrates 4a and 4b can be accurately detected.

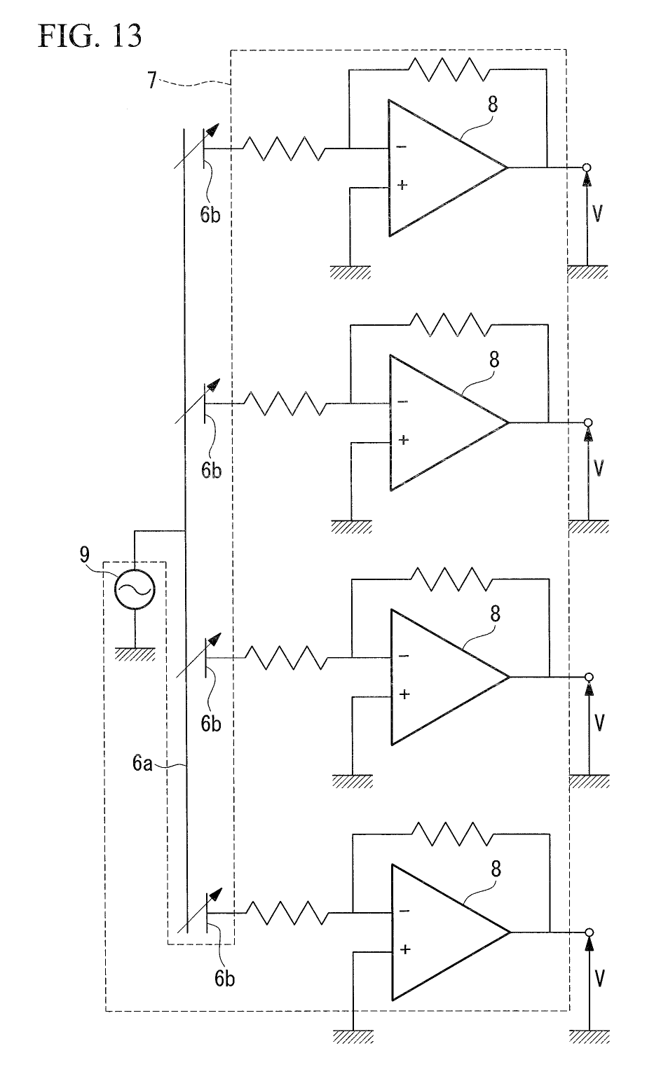

The endoscope system 10 according to this embodiment may employ the variable spectroscopy element 1 shown in any one of FIGS. 1 to 8. For example, if the variable spectroscopy element 1 shown in FIG. 7 is to be employed, the electrical circuit 7 shown in FIG. 13 may be employed.

The electrical circuit 7 employed is a circuit that detects the capacitance as an electrical signal and amplifies it. However, the present invention is not limited to such a configuration and may alternatively employ a buffer circuit not having an amplifying function. An example of a buffer circuit is a voltage follower circuit. With the buffer circuit, the output impedance of a sensor output can also be reduced so that noise immunity can be improved.

The endoscope system 10 according to this embodiment described above is a system configured to acquire an agent-fluorescence image and a reflection image. Alternatively, the present invention can be used for acquiring a combination of other images, such as an autofluorescence image and an agent-fluorescence image or an autofluorescence image and a reflection image.

In this embodiment, a circuit that converts a capacitance value to a voltage value is used as the electrical circuit 7 for the sensors 6. Alternatively, a circuit that converts a capacitance value to a current value may be used as the electrical circuit 7.

In this embodiment, the endoscope system 10 having the bending portion 11b is described as an example. Alternatively, application to a rigid borescope not having the bending portion 11b or application to a capsule endoscope is also permissible. Furthermore, the observation object A is not limited to a living organism. The present invention can be applied to an industrial endoscope intended for an interior of a pipe, a machine, a structure, etc.

In this embodiment, the endoscope system 10 described above includes the variable spectroscopy element 1 provided in the image-acquisition unit 2. Alternatively, the variable spectroscopy element 1 may be provided in a light source unit 30 disposed at the tip of the insertion section 11.

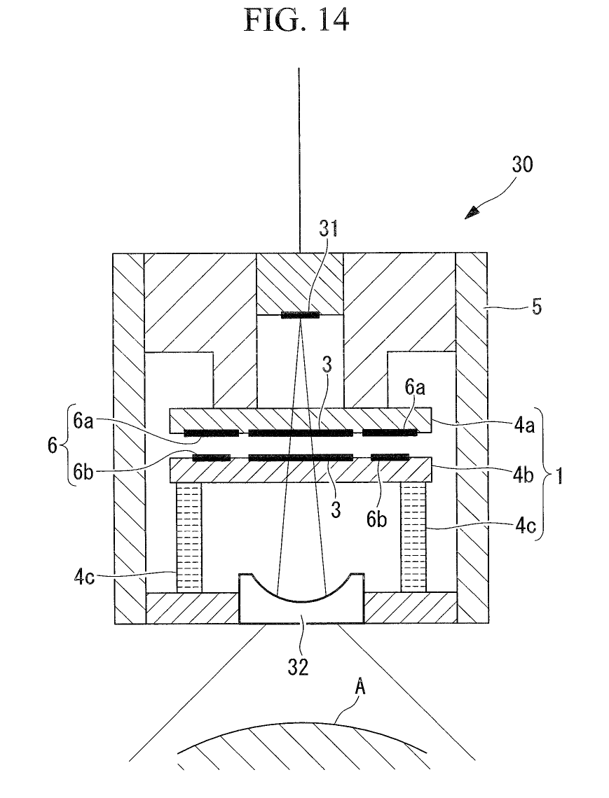

As shown in FIG. 14, the light source unit 30 includes a white LED (photoelectric conversion element) 31 that generates white light, the aforementioned variable spectroscopy element 1, a lens 32 that expands the white light emitted from the white LED 31, and the frame member 5 that supports these components.

Accordingly, even if the optical substrates 4a and 4b become relatively displaced in the direction perpendicular to the optical axis when the actuators 4c of the variable spectroscopy element 1 are driven, there is no change in the value of the capacitance detected by the sensors 6, and the spacing between the optical substrates 4a and 4b can be accurately detected, whereby illumination light in a certain wavelength range accurately split off from the white light can be emitted to the observation object A.

As an alternative to the case where a single white LED 31 is provided, the light source unit 30 may be provided with a plurality of white LEDs 31 in order to increase the amount of illumination light and to improve the light distribution characteristics. As another alternative, the light-source area may be increased by using a combination of a single white LED 31 and a diffuser panel, or a lamp etc. may be used.

As a further alternative, a semiconductor laser of a multi-wavelength excitation type or a super-luminescent diode, for example, may be used.

1. A variable spectroscopy element comprising:
optical coatings provided on opposing surfaces of first and second optical substrates that face each other with a spacing therebetween;
an actuator that adjusts the spacing between the first and second optical substrates;
a first sensor electrode that detects the spacing between the first and second optical substrates and is provided on the first optical substrate; and
a second sensor electrode that detects the spacing between the first and second optical substrates, the second sensor electrode facing the first sensor electrode and provided within a region of the second optical substrate onto which the first sensor electrode is projected.

2. The variable spectroscopy element according to claim 1, wherein the first and second sensor electrodes have a similar shape.

3. The variable spectroscopy element according to claim 2, wherein the first and second sensor electrodes are circular.

4. The variable spectroscopy element according to claim 1, wherein the optical coatings are composed of a conductive material, and
wherein the first and second sensor electrodes are formed of the optical coatings.

5. The variable spectroscopy element according to claim 1, wherein the first and second sensor electrodes have different shapes.

6. The variable spectroscopy element according to claim 1, wherein the first and second sensor electrodes have a dimensional difference that is greater in a circumferential direction than in a radial direction.

7. The variable spectroscopy element according to claim 1, wherein the optical coatings transmit light of a desired wavelength range.

8. A spectroscopy apparatus comprising:
a variable spectroscopy element according to claim 1, and
an image-acquisition unit that acquires an image of light split by the variable spectroscopy element.

9. An endoscope system comprising the spectroscopy apparatus according to claim 8.
